(19) United States
(12) Patent Application Publication
(10) Pub. No.: US 2021/0121419 A1
(43) Pub. Date: Apr. 29, 2021

(54) METFORMIN COMPOUNDED COMPOSITION AND METHOD THEREOF

(71) Applicant: ZHOUSHAN TRITECH BIOSCIENCES, INC., ZHOUSHAN (CN)

(72) Inventor: Xinli LIN, SHENZHEN (CN)

(21) Appl. No.: 17/140,089

(22) Filed: Jan. 3, 2021

Related U.S. Application Data
(63) Continuation of application No. PCT/CN2019/097801, filed on Jul. 26, 2019.

(30) Foreign Application Priority Data
Jul. 27, 2018 (CN) 201810839940.8

(51) Int. Cl. A61K 31/155; A61P 3/06; A61P 9/12; A61P 27/04; A61P 27/02; A61P 1/10; A61K 31/202; A61K 31/593; A61K 31/455; A61K 31/355; A61K 36/062; A61K 9/00; A61K 36/899

(52) U.S. Cl. CPC A61K 31/155; A61P 3/06; A61P 9/12; A61P 27/04; A61P 27/02; A61P 1/10; A61K 36/899; A61K 31/593; A61K 31/455; A61K 31/355; A61K 36/062; A61K 9/0053; A61K 31/202

(57) ABSTRACT

The present disclosure is related to a metformin compounded composition and application thereof. The disclosure provides active ingredients of composition dosage forms, comprising active ingredients including metformin, omega-3 fatty acid, statin, and vitamins. The whole or part of the active ingredients can be manufactured into a single dosage form for preventing or treating a special disease or enhance health in general. The diseases that can be prevented or treated include cardiovascular diseases, nervous system diseases, cancer, diabetes, autoimmune diseases, or any other diseases that can be treated with the provided formulations. Because of the broad applicability for preventing diseases and enhancing health, the application of the invented formulation may provide “health and longevity” for general population.

### CROSS-REFERENCE TO RELATED APPLICATIONS

This application is a continuation of International Patent Application No. PCT/CN2019/097801 with a filing date of Jul. 26, 2019, designating the United States, now pending, and further claims priority to Chinese Patent Application No. 201810839940.8 with a filing date of Jul. 27, 2018. The content of the aforementioned applications, including any intervening amendments thereto, are incorporated herein by reference.

### TECHNICAL FIELD

The present disclosure is related to the field of pharmaceuticals and dietary supplements. The disclosure provides active ingredients comprising compounded composition including metformin, omega-3 fatty acids, statin, and vitamins, for enhancing health in general and for preventing and treating common and frequently-encountered diseases, such as diabetes, cardiovascular diseases, inflammatory, autoimmune diseases, cancer, eye diseases, central or peripheral nervous system diseases, and intestinal diseases such as constipation.

### BACKGROUND

Metformin is a classic medicine to treat type 2 diabetes, and have been confirmed by the International Diabetes Federation as a “cornerstone for treating type 2 diabetes medicine”. The safety and efficacy of metformin have been proved by large scale clinical application over 60 years. In 1998, The UK Prospective Diabetes Study (UKPDS) has made a landmark decision to affirmed the long-term protective effect of metformin for cardiovascular system: “metformin is the only hyperglycemic medicine that also decreases cardiovascular complications, and also can decrease type 2 diabetes complication and mortality”. Furthermore, because of the general effect for preventing and treating diseases and enhancing health, and the longevity effect proven by in clinical settings, the US FDA has approved the first anti-aging clinical trials using metformin. The clinical trials may turn the now common diseases such as cardiovascular diseases, diabetes, cancer, Alzheimer's and Parkinson diseases into rare conditions.

Omega-3 fatty acids are approved drug and also a common health supplement, considered to be homology of medicine and food. Omega-3 fatty acids have broad effect for keeping people stay healthy, and can prevent and treat diseases. The US FDA has approved three Omega-3 fatty acid drugs, for treating hereditary hypertriglyceridemia, and for treating other patients with acquired hypertriglyceridemia. Omega-3 fatty acids also have broad health benefits, include regulating immune system and treating nervous system diseases.

Statin drugs are currently the most broadly used lipid-lowering drugs clinically, and are inhibitors of HMG-CoA (3-hydroxy-3-methylglutaryl-coenzyme A) reductase, which is the rate limiting enzyme in the cells for cholesterol synthesis. Statins can reduce free cholesterol in cells, decrease low density lipoprotein (LDL) and very low density lipoprotein (VLDL) in the blood. As a result, statins can significantly lower cholesterol and LDL to reach the goal of reducing blood lipid. The main function of statin is to lower the level of LDL-cholesterol. It has been proved that statin can reduce the risk of cardiovascular diseases by half. In July 2015 Harvard School of Public Health suggested larger-scale usage of statins. Large-scale research in the US has indicated that if 48-67% of 40-75 years old are using statin, it will result in lowering medical cost of the nation, and at the same time, to avoid more than 160,000 cardiovascular diseases in the US. If statin drugs and supplements can receive large-scale application in China, it will certainly greatly enhance the general health of the Chinese people, and at the same time, greatly reducing health cost as a whole.

Vitamin D has broad health effect for human body, including regulating immune system, preventing cardiovascular diseases, cancer, and Alzheimer's disease caused by chronic inflammation.

Vitamin B is essential for promoting metabolism, to convert sugar, fat, proteins into energy. In addition, research has shown that Vitamin B3 (Niacin) can lower cholesterol and triglyceride, and have anti-aging effect.

At present, metformin, Omega-3 fatty acids, and statin are all in single dosage form, which has limitations in enhancing health effect. There has no compounded composition dosage form including the combination of metformin, Omega-3 fatty acids, and statins in the present technology.

### SUMMARY OF THIS DISCLOSURE

The present disclosure provides a compounded composition comprising metformin, Omega-3 fatty acids, and statin compounds. The compounded composition dosage forms can solve the problems of limitations when a single dosage form is used for enhancing health.

The practical technical solution of the present disclosure is: providing a metformin compounded composition, in which the active ingredient of the metformin compounded composition comprises metformin, Omega-3 fatty acids, statin, and vitamins.

In one embodiment, the aforementioned metformin compounded composition comprises, by weight ratio: metformin:Omegs-3 fatty acids:statin=50-500:1000:1-300.

In another embodiment, the aforementioned metformin compounded composition comprises, by weight ratio: metformin:Omegs-3 fatty acids:statin=100-300:1000:1-10.

In yet another embodiment, in the aforementioned metformin compounded composition, the said vitamins comprise vitamin D and/or vitamin B and/or vitamin E. Optimally, the said vitamin D is vitamin D3, the said vitamin B is vitamin B3.

In yet another embodiment, in the aforementioned metformin compounded composition, the said Omega-3 fatty acids comprise EPA and/or DHA. Optimally, the said Omega-3 fatty acids are ethyl, triglyceride, or free acid form of EPA and/or DHA; or the said Omega-3 fatty acids comprise oily form and dry powder forms of EPA and/or DHA; or the said Omega-3 fatty acids comprise EPA and/or DHA salt, and the said salt includes sodium, potassium, calcium or any other edible salts.

In yet another embodiment, in the aforementioned metformin compounded composition, the said statin comprises chemically synthesized drugs or statin-containing natural products.

In yet another embodiment, in the aforementioned metformin compounded composition, the said statin-containing natural product is red yeast rice.

The present disclosure comprises a medicine comprises metformin compounded composition in a dosage form, wherein the medicine is produced into an oral dosage form.

Furthermore, in the aforementioned metformin compounded composition, the said oral dosage form is to make an oral capsule or pill, containing 0.1 mg to 2 g of the aforementioned metformin compounded composition; or to make oral capsule or pill containing 0.1 mg to 2 g of individual active ingredient of the aforementioned metformin compounded composition, to take orally at the same time or different times.

The application of the aforementioned metformin compounded composition for preventing and/or treating cardiovascular diseases, diabetes, chronic inflammation, cancer, autoimmune disease, eye disease, nervous system disease, and constipation.

The metformin compounded composition of the present disclosure, formulated in certain scale, can have synergetic enhancement effect for preventing and treating diseases and enhance health generally, and can promote general “health enhancement and longevity” with long-term usage.

Metformin is an essential drug for treating type-2 diabetes, but has been find to have anti-aging and health-promoting effect in extensive animal tests and long-term epidemiologic studies. Research findings indicated that other than therapeutic effect for type-2 diabetes through regulating metabolism, metformin can also decrease age-related cardiovascular diseases, cancer, autoimmune diseases, nervous system diseases, and intestinal diseases such as constipation through regulating basic metabolism and intestinal microbiome.

Omega-3 fatty acids, comprising the health-promoting “active” ingredient of all-cis-5,8,11,14,17-eicosapentaenoic acid (EPA, chemical structure shown in FIG. 1), and all-cis-4,7,10,13,16,19-docosahexaenoic acid (DHA, chemical structure shown in FIG. 2), are required for a normal, healthy life. However, they cannot be synthesized by the body and must be obtained from food or dietary supplements sources, and therefore are termed essential fatty acids. The chemical formulations of EPA and DHA are shown below:

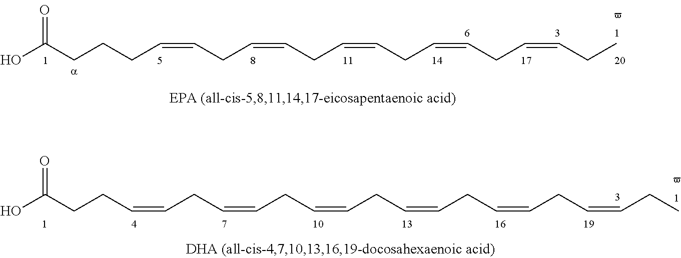 [SMILES: CC/C=C\C/C=C\C/C=C\C/C=C\C/C=C\CCCC(=O)O ; CC/C=C\C/C=C\C/C=C\C/C=C\C/C=C\C/C=C\CCC(=O)O]

Large population studies have shown that omega-3 fatty acids (EPA and DHA) can reduce triglycerides, heart rate, blood pressure, and atherosclerosis. Omega-3 fatty acids (EPA and DHA) may act as direct ligands to a cell surface G-protein receptor, affecting antiinflammatory and insulin sensitization, and thus can be used to treat inflammation caused illnesses and diabetes. It has been shown that rheumatoid arthritis sufferers taking omega-3 fatty acids from sources such as fish have reduced pain. Omega-3 fatty acids (EPA and DHA) may also be used to alleviate neurological syndromes such as depression and Alzheimer's disease. Omega-3 can delay or prevent the progression of certain psychotic disorders such as schizophrenia. Omega-3 fatty acids (EPA and DHA) may also reduce the risk of thromboembolism related illness such as ischemic and thrombotic stroke Omega-3 fatty acids (EPA and DHA) may also have anti-cancer effects, in particular breast, colon, and prostate cancer. Clinical evidence showed that Omega-3 fatty acids can also be used to treat xerophthalmia and muscae volitantes from unknown causes.

Statins are the most widely used lipid (low density lipoprotein, LDL cholesterol) lowering drugs. Because of its broad health promoting effects, the Harvard School of Public Health suggested wider-scale usage of statins in 2015. Specifically, it recommends 48-67% of 40-75 years old Americans to use statins to reduce cholesterol, and estimate that every year more than 160,000 cardiovascular diseases, including heart diseases, myocardial infarction, and strokes, can be avoided in the US if practiced.

Statin containing compounds comprise chemical drugs contain a single statin, including natural statin compound (such as lovastatin, simvastatin, pravastatin, and mevastatin) and chemically synthesized statin (such as Fluvastatin, atorvastatin, cerivastatin, rosuvastatin, and pitavastatin). These types of drugs are the most classic and effective lipid lowering drugs, broadly used for the treatment of hyperlipidemia and hypertension.

Statin containing compounds also comprise natural products such as red yeast rice. Red yeast rice (Red Yeast) contains natural statin as its main active ingredient. Red yeast rice was first discovered in China, and has been used in traditional Chinese herbology and traditional Chinese medicine for more than a thousand years. Red yeast rice is traditional fermentation product of rice in China and surrounding countries. It is taken orally to invigorate the body, aid in digestion, and revitalize the blood, according to the theory of traditional Chinese medicine

Modern biologist isolated new Monascus from many traditional strains, and find natural statin compounds that can lower serum cholesterol. After large amount of screening and selection, they made mutated strain suitable for industrial production, which can produce high potency, stable, and suitable to manufacture medical drug. The new strain not only can make natural statin-type compounds that can inhibit a critical enzyme in cholesterol synthesis, the HMG-CoA (3-hydroxy-3-methylglutaryl-coenzyme A) reductase, but can also produce many compounds beneficial to human health, such as essential amino acids, unsaturated fatty acids etc. Large number of studies have found that red yeast rice has very powerful comprehensive effect to lower total cholesterol, lower low density lipoprotein cholesterol, to lower hardening index of atherosclerosis, and raise high density lipoprotein cholesterol, in addition to its high safety, low side effect. Red yeast rice can be effectively used to treat cardiovascular diseases such as coronary heart disease and stroke, in addition to hyperlipidemia related diseases such as diabetes, kidney disease, and fatty liver disease.

Vitamins are essential for human body, but cannot be synthesized in vivo, or cannot be made enough in vivo, and must be obtained by food or supplements. Vitamins are essential for human metabolism, growth, development and health. Long-term deficient of certain vitamins can result in physiological dysfunctions sickness.

There are dozens of essential vitamins discovered currently, including vitamin A, vitamin B, vitamin C, and vitamin D, etc. Vitamins can be obtained from food, but can also generally be obtained from supplements. In imbalanced diet, and certain food adsorption disorders, it becomes essential to take extra purified supplemental vitamins. In many cases, the deficiencies of vitamin B and vitamin D are unconsciously threatening human health.

Vitamin B is an essential substance in promoting in vivo metabolism, converting energy sources such as sugar, fat, and proteins into heat. Furthermore, research indicated that vitamin B3 (niacin) can lower cholesterol and triglyceride, and has anti-aging effect. Hence supplementing vitamin B, in addition to Omega-3 fatty acids and statin in the compounded composition herein have synergistic effect in keeping cardiovascular health.

Initially it was discovered that Vitamin D's biological function is to help the body to absorb phosphate and calcium, and vitamin D is an essential element for bone health because vitamin D deficiency can result in rickets disease. But modern researches showed that Vitamin D has broader biological functions, including regulating immune system, preventing cardiovascular diseases, cancer, and Alzheimer's disease, etc, all can be caused by chronic inflammation. Because of diet imbalance, and very little sun exposure, most of the modern human have “sub-health” conditions caused by Vitamin D deficiency. Big data analysis showed the levels of Vitamin D in about 60-80% of Asian population are lower than required, therefore supplement Vitamin D can enhance health in general. Vitamin D in said dosage form can enhance health and prevent diseases through synergistic effect with other active ingredients.

The formulation of the present disclosure is a composition comprises metformin, Lovaza-type Omega-3 fatty acids, statin-containing compounds including red yeast rice, and vitamins. Through regulation of enteric microorganism, metformin can prevent and treat diseases, enhance health and increasing life-span through delaying intestinal absorption of glucose, and increasing glucose utilization through increasing insulin sensitivity. The combination of Omega-3 and statin on one hand reserved the triglyceride-lowering effect and overcome the shortcoming of increasing LDL-C of Lovaza, at the same time reserved and enhanced the long-term effect in preventing and treating cancer and nervous system disease including Alzheimer's disease of the DHA in Lovaza Omega-3 fatty acid. The principle of overcoming side effects includes several aspects. The first is that statin drugs (including red yeast rice) can overcome the side effect of increasing LDL-C level of Lovaza, at the same time amplify its lipid-lowering effect. The second is when the compounded composition is used, the dosage for individual component can be decreased to reach the same therapeutic efficacy. Lower individual dosage can greatly decrease even eliminated side effects. Third, the compounded composition can reach the same therapeutic efficacy of Amarin's pure EPA drug Vascepa (reduce triglyceride but no increase LDL-C), but the advantage is that the formulation can reserve all of the DHA's health-enhancement effects in Lovaza, including immune-regulation and prevention and treatment effect for nervous system. The health benefit for nervous system from DHA in Omega-3 has been proven in many clinical experiments, and the pivotal role in infant's nerve grow has also been formed consensus in the field. Currently, enough DHA (100-300 mg/dose) is contained in most of the supplement for pregnant women, baby formula, and child multiple vitamins. Fourth, statin drugs have no or very little effect for about ⅕ of hypertension patients, and the compounded composition can provide new choice for these population, which can be effective for a large portion of the patients.

The present formulation also provides composition of vitamins, comprising one type or multi-type vitamins.

The present disclosure also provides preparation methods of the invented compounded composition. The preparation methods are known to those skill in the art.

The present disclosure combined the beneficial effects of metformin, statin drugs, vitamins, and Omega-3 fatty acids, and decreased or eliminated shortcomings or side effects in individual dosage, to prepare the individual active ingredients into a compounded composition dosage form, for enhance health effects. The present composition can also be formulated into medical drug dosage to treat various diseases, including but not limited to inflammation, nervous system diseases, cancer, eye disease, especially cardiovascular system and related diseases. Furthermore, the effects of the compounded composition also include enhance and stay healthy and promote longevity.

In one embodiment, the present disclosure provides functional dietary supplemental or pharmaceutical compounded composition to enhance health and therapeutic effect of the individual ingredient. The combination formula of metformin, statins, vitamins, and Omega-3 fatty acids can have synergetic enhancement effect in preventing and treating cardiovascular system and related illness.

Further in another embodiment, the present disclosure provides functional dietary supplemental or pharmaceutical compounded composition comprising various ratios of metformin, statin, vitamins, and Omega-3 fatty acids, and other active ingredients that can enhance the health of cardiovascular system such as antioxidants. These active ingredients in the present composition can synergistically enhance the individual ingredients' effects for promoting health and treating diseases.

In a related embodiment, Omega-3 fatty acids comprise about 5-95% EPA fatty acid or its derivatives, such as EPA-Ethyl Ester by weight. In yet another related embodiment, Omega-3 fatty acids comprise about 5-95% DHA fatty acid or its derivatives, such as DHA-Ethyl Ester by weight. Further in yet another embodiment, Omega-3 fatty acids comprise mixtures of about 5-95% EPA and DHA fatty acid or its derivatives, such as DHA-Ethyl Ester by weight.

Further in another embodiment, the present disclosure provides a method to enhance health and longevity using functional supplement. The method comprises to provide to the desired object the composition or compounded composition of the present disclosure.

Further in yet another embodiment, the present disclosure provides a method to treat cardiovascular system and related diseases. The method comprises to provide to the desired object the composition or compounded composition of the present disclosure. In one embodiment, the cardiovascular disease is arteriosclerosis.

Further in yet another embodiment, the present disclosure provides a method to treat eye diseases. The method comprises to provide to the desired object the composition or compounded composition of the present disclosure. In one embodiment, the eye disease includes but not limited to muscae volitantes, xerophthalmia, etc.

Further in yet another embodiment, the present disclosure provides a method to treat inflammation, autoimmune disease, cancer, and nervous system related diseases. The method comprises to provide to the desired object the composition or compounded composition of the present disclosure.

As used herein, metformin is a chemical drug, with its English name as Metformin, chemical name as 1,1-Dimethylbiguanide.

As used herein, “EPA” is all-cis-5,8,11,14,17-eicosapentaenoic acid, and “DHA” is all-cis-4,7,10,13,16,19-docosahexaenoic acid. The terms EPA and DHA are used to indicate both the triglyceride and esterized forms of these fatty acids unless the triglyceride or esterized form is clearly indicated by the context. In particular, the esterized form includes EPA ethyl ester (ethyl-EPA) and DHA ethyl ester (ethyl-DHA). It is clearly understood that EPA and DHA also include all forms of pharmaceutically acceptable fatty acid salts.

As used herein, an Omega-3 fatty acid includes all physical forms of omega-3 fatty acids, such as in oil form, dry powder form, and any forms in between.

As used herein, an omega-3 fatty acid includes all forms of “active” omega-3 fatty acids, such as EPA and DHA or derivatives thereof, for example ethyl-EPA and ethyl-DHA. An omega-3 fatty acid also includes unpurified or natural forms of the acid such as naturally present in deep-sea fish oil and other oils, and purified forms such as purified EPA and DHA and mixtures thereof.

As used herein, vitamins include all known vitamins, especially vitamin B and vitamin D.

As used herein, a “dosage form” or a “dosage” means administration of a unit of active medicine. Active ingredients comprising various combination of metformin, omega-3 fatty acid, statin containing compounds including red yeast rice, and vitamins. Examples of dosage forms include tablets, capsules (particularly gel and liquid capsules including enteric softgel capsules), suspensions, liquids, candy and chewable formulations, emulsions, creams, ointments, suppositories, and the like. “Gel capsule” means any soft gelatin, liquid-filled capsule that contains a liquid, liquid suspension, solution, gel, or emulsion.

As used herein, the major active ingredients of the compounded composition in the present disclosure comprise metformin, Omega-3 fatty acids, statin-containing compounds including red yeast rice and vitamins.

As used herein, metformin, Omega-3 fatty acids, statin containing compounds including red yeast rice, and vitamins including vitamin E are all individually and collectively called active ingredients.

As used herein, the term “cardiovascular related diseases” refers to any diseases or disorders of the heart or blood vessels (i.e. arteries and veins) or any symptom thereof herein. Non-limiting examples of cardiovascular-related diseases include hypertriglyceridemia, hypercholesterolemia, mixed dyslipidemia, coronary heart disease, vascular disease, stroke, atherosclerosis, arrhythmia, hypertension, myocardial infarction, and other cardiovascular events.

As used herein, the term “prevention” in relation to a given disease or disorder means: preventing the onset of disease development if none had occurred, preventing the disease or disorder from occurring in a subject that may be predisposed to the disorder or disease but has not yet been diagnosed as having the disorder or disease, and/or preventing further disease/disorder development if already present. The term “treatment” in relation a given disease or disorder, includes, but is not limited to, inhibiting the disease or disorder, for example, arresting the development of the disease or disorder; relieving the disease or disorder, for example, causing regression of the disease or disorder; or relieving a condition caused by or resulting from the disease or disorder, for example, relieving, preventing or treating symptoms of the disease or disorder.

As used herein, statin or statin containing compounds comprise chemical drugs that may contain a single stain, including natural statin compound (such as lovastatin, simvastatin, pravastatin, and mevastatin) and chemically synthesized statin (such as Fluvastatin, atorvastatin, cerivastatin, rosuvastatin, and pitavastatin), and/or compounded statin drugs; red yeast rice comprises all forms of red yeast rice, including dry powder form, tablets and capsules, etc. Red yeast rice can be produced by traditional Chinese methods, and can also be produced by modern isolated Monascus strains.

As used herein, vitamins comprise all forms of vitamins, including water soluble and liposoluble forms.

The present disclosure also provides dosage forms of such formulations comprising about 50-5000 mg of metformin, about 50-1000 mg of DHA and about 50-1000 mg of EPA, about 1-30 mg of statin, and about 0.0001-100 mg of vitamin. Various combinations of the active ingredients can be manufactured in a unit dosage form.

Although the present disclosure is capable of being embodied in various forms, the description of various embodiments is made with the understanding that the present disclosure is to be considered as an exemplification of the disclosure, and is not intended to limit the disclosure to the specific embodiments illustrated.

In one embodiment, the present disclosure provides a dietary supplemental or pharmaceutical formulation comprising metformin, statin, vitamins, and omega-3 fatty acids.

In various embodiments, certain amounts of metformin, statin-containing compound, vitamins, omega-3 fatty acids, and other auxiliary ingredients is formulated into an oral unit dose. Each component or active ingredient in the dose may be generally varied or adjusted.

In various embodiments, one of the active ingredients in a formulation is metformin. In certain embodiment, metformin is a metformin hydrochloride salt.

In various embodiments, the amount of metformin in a unit oral dosage can generally be varied. Metformin can be used in long-term in a “low dose”, but effective dosage. “Low dose” is in an amount of 50-500 mg per day. Normal dosage can also be used to reach better therapeutic efficacy, such as 500-5000 mg per day.

In various embodiments, the amounts of metformin in an oral dosage unit dose can generally be varied. The adjustable range can be from about 50 mg to about 5000 mg, from about 75 mg to about 4000 mg, from about 100 mg to about 3000 mg, from about 200 mg to about 2000 mg, from about 300 mg to about 1000 mg, from about 400 mg to about 800 mg, from about 500 mg to about 600 mg, from about 300 mg to about 500 mg, from about 100 mg to about 300 mg, about 100 mg, about 200 mg, about 300 mg, about 400 mg, about 500 mg, about 600 mg, about 700 mg, about 800 mg, about 1000 mg, about 2000 mg, about 3000 mg, about 4000 mg, about 5000 mg.

One of the new discoveries of the present disclosure is the important health effect of low dose metformin. The normal dosage of metformin in Chinese population is 1-2 g per day, and the dose for the anti-aging clinical trial in the US is 1.7 g per day. It is known that one of the functions of metformin in regulating human health is through regulating enteric microorganism, whereas an unexpected discovery of the present disclosure is that low dose metformin (0.1-0.5 g/day, prefer for 0.125-0.25 g/day) can also promote health through regulating enteric microorganism. The application of low dose metformin can avoid long-term application caused side effects such as hypoglycemia, lactic acidosis, and hepatic and renal toxicity when normal dosage is used. The application in a large population showed that 2-3 days after low dose metformin (0.125-0.25 g/day) usage, mild diarrhea occurs, but stool returned to normal in 4-5 days, apparently showed that the low metformin dosage caused readjustment and regrouping of enteric microorganism. Clinical trials have proved that readjusted and regrouped enteric microorganism by metformin can have health effects by preventing and treating diseases, and have longevity effect for human life span.

In various embodiments, one active ingredient of the formulation comprises an omega-3 fatty acid. In another embodiment, the omega-3 fatty acid comprises EPA or DHA or a pharmaceutically acceptable ester-form, triglyceride-form, other derivative-form, conjugate or salt thereof, or mixtures of any of the foregoing.

In various embodiments, the amounts of omega-3 fatty acids in an oral unit dosage form for a patient may be generally varied or adjusted from about 50 mg to about 2000 mg, from about 75 mg to about 1500 mg, from about 100 mg to about 1200 mg, from about 200 mg to about 1000 mg, from about 300 mg to about 800 mg, from about 300 mg to about 500 mg, about 100 mg, about 200 mg, about 300 mg, about 400 mg, about 500 mg, about 600 mg, about 700 mg, about 800 mg, about 1000 mg.

In various embodiments, one of the active ingredients of the formulation comprises statin.

In various embodiments, the amount of statin in an oral unit dosage for a patient may be generally varied, and can be adjusted from about 0.1 mg to about 300 mg, from about 1 mg to about 300 mg, from about 1 mg to about 100 mg, from about 1 mg to about 10 mg, from about 5 mg to about 75 mg, from about 10 mg to about 50 mg, from about 15 mg to about 40 mg, about 3 mg, about 5 mg, about 6 mg, about 9 mg, about 10 mg, about 15 mg, about 20 mg, about 25 mg, about 30 mg, about 35 mg, about 40 mg.

In various embodiments, one of the active ingredients of the formulation comprises red yeast rice. The natural statin in red yeast rice can be about 0.01-5%. Different statin content has different therapeutic effect and impact to health.

In various embodiments, the amount of red yeast rice in an oral unit dose form for a patient may be generally varied or adjusted from about 1 mg to about 10 mg, from about 50 mg to about 1000 mg, from about 75 mg to about 750 mg, from about 125 mg to about 500 mg, from about 200 mg to about 400 mg, about 100 mg, about 200 mg, about 300 mg, about 400 mg, about 500 mg, about 600 mg, about 700 mg, about 800 mg.

In various embodiments, one active ingredient of the formulation comprises vitamins.

In various embodiments, the amounts of vitamins in an oral unit dose form for a patient may be generally varied or adjusted from about 0.0001 mg to about 100 mg, from about 0.001 mg to about 10 mg, from about 0.01 mg to about 5 mg, from about 0.05 mg to about 1 mg, about 0.01 mg, about 0.1 mg, about 0.001 mg, about 0.01 mg, about 0.1 mg, about 0.5 mg, about 1 mg, about 5 mg, about 10 mg, about 20 mg, about 50 mg, about 100 mg.

The composition provided herein may be provided to a patient in any numbers of pharmaceutically acceptable oral dosage forms. Preferably the composition dosage can be orally administered in the form of pills, tablets, or gel capsules or the like. The composition can also be prepared into convenient orally available forms such as drinks and candies. However, the administration could also be through any other route where the active ingredients may be efficiently absorbed and utilized, e.g. intravenously or subcutaneously. Also included herein are pharmaceutical compositions, comprising pharmaceutical formulations in a unit dosage form. In such dosage forms, the formulation is subdivided into suitably sized unit doses containing appropriate quantities of the active ingredients in the composition, with an effective amount to achieve the desired purpose.

The present disclosure provides capsule (hard capsule and softgel, etc), tablet, liquid, syrup, suspensions, sublingual, candy, and chewable dosage forms of the compounded composition accordingly.

The present disclosure also includes methods for making pharmaceutical compositions comprising the compounded compositions described herein.

Pharmaceutical dosage forms may contain excipients. Excipients must be of commonly used low toxicity and sufficiently high purity to render them suitable for administration to a patient being treated.

Excipients are selected with respect to the intended form of administration, e.g. oral tablets, capsules, powders, syrups, suspensions, and the like, and consistent with conventional pharmaceutical practices, and are well known for those with skill in the art. For example, for oral administration in the form of gel capsule the composition formulation may be combined with a preservative, flavorant, colorant or the like.

The present disclosure includes solid dosage forms such as tablets and capsules. A capsule may be prepared by placing the compounded composition, described herein, inside a capsule shell. A capsule is a dosage form administered in a special container or enclosure containing the active ingredients. In some embodiments the composition active ingredients plus excipients are filled into hard or soft capsules. A capsule shell may be made of methylcellulose, hydroxypropylmethyl cellulose, polyvinyl alcohols, or denatured gelatins or starch or other material. Hard shell capsules are typically made of blends of relatively high gel strength bone and pork skin gelatins. In some embodiments the unit dosage form is a gel capsule. In some embodiments the capsule shell is a glycerin capsule shell. In other embodiments the capsule is a bovine gelatin shell. Other suitable capsule shell materials include polyethylene, polypropylene, poly(methylmethacrylate), polyvinylchloride, polystyrene, polyurethanes, polytetrafluoroethylene, nylons, polyformaldehydes, polyesters, cellulose acetate, and nitrocellulose. The capsule shell itself may contain small amounts of dyes, opaquing agents, plasticizers, and preservatives. Conventional methods for preparing other solid dosage forms, for example, capsules, suppositories, and the like are also well known. Gelatin capsule shells may be made also be made of tapioca, grass, vegetable derived or fish derived gelatin. Other vegetarian derived gelatin capsules may be made of vegetable derived hydroxypropylmethyl cellulose (HPMC). Capsules shells may also contain Modified Maize Starch, Glycerol, and Carrageenan as a gelling agent.

In other embodiments the capsule has a shell comprising the material of the rate-limiting membrane, including coating materials, and filled with compounded composition active ingredients of the present disclosure. Capsule shells may be composed of a porous or a pH-sensitive polymer made by a thermal forming process. In certain embodiments the capsule shell is in the form of an asymmetric membrane; i.e., a membrane that has a thin skin on one surface and most of whose thickness is constituted of a highly permeable porous material. In certain embodiment the active ingredients of the said compounded composition are filled into an enteric coated soft gel or capsule with a standard and well-known manufacture process.

In certain embodiments the active ingredients of the present disclosure are filled into a slow-release or/and extended-release capsule for timely and steady release of the active ingredients. Various technologies for slow-release or/and extended-release formulations are well known for those with skill in the art.

An example of a useful enteric capsule, a “swelling plug device”, can be used. The active ingredients of the said composition of the present disclosure can be incorporated into a non-dissolving capsule-half of the device which is sealed at one end by a hydrogel plug. This hydrogel plug swells in an aqueous environment, and, after swelling for a predetermined time, exits the capsule, thus opening a port through which the active agent can leave the capsule and be delivered to the aqueous environment. Preferred hydrogel-plugged capsules are those which exhibit substantially no release of active agent from the dosage form until the dosage form has exited the stomach and has resided in the small intestine for about 15 minutes or more, preferably about 30 minutes or more, thus assuring that minimal omega-3 plus active ingredients are released in the stomach.

Conventional methods for preparing tablets are well known in the field. Such methods include spray drying methods such as direct compression and compression of granulation produced by compaction, or wet methods or other special procedures.

Liquid form preparations include solutions, suspensions and emulsions. Examples of liquid pharmaceutical preparations include propylene glycol solutions and solutions containing sweeteners for oral solutions, suspensions and emulsions.

The said composition formulation dosage form can be contained in a plasticizer, particularly in a capsule shell. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose, and triacetin.

The said composition formulation dosage form described herein may be coated. The coating can be an enteric coating, i.e. a coating that is predominantly soluble in the intestinal fluid, but substantially insoluble in the gastric fluids. Examples of coating materials included polyvinyl acetate phthalate (PVAP), commercially available under trade names of Opadry® Enteric from Colorcon® hydroxypropylmethylcellulose acetate succinate (HPMCAS), cellulose acetate phthalate (CAP), methacrylic acid copolymer, hydroxypropylmethylcellulose succinate, cellulose acetate succinate, cellulose acetate hexahydrophthalate, hydroxypropylmethylcellulose hexahydrophthalate, hydroxypropylmethylcellulose phthalate (HPMCP), cellulose propionate phthalate, cellulose acetate maleate, cellulose acetate trimellitate, cellulose acetate butyrate, cellulose acetate propionate, methacrylic acid/methacrylate polymer, methacrylic acid-methyl methacrylate copolymer, ethyl methacrylate-methylmethacrylate-chlorotrimethylammonium ethyl methacrylate copolymer, and the like, and combinations comprising one or more of the foregoing enteric polymers. Other examples include natural resins, such as shellac, SANDARAC, copal collophorium, and combinations comprising one or more of the foregoing polymers. Yet another set of examples of enteric polymers include synthetic resin bearing carboxyl groups. The methacrylic acid:acrylic acid ethyl ester copolymers are commercially available under the trade names of “Eudragit® L”, such as Eudragit® L 30-D55 from Degussa.

The composition formulation described herein may include a stabilizer. “Stabilizers” include compounds which maintain a desirable attribute of the formulation over a time interval including but not limited to mechanical, chemical and temperature stressing that can be tested in a laboratory setting. Such attributes include stabilizing homogeneity, resulting in concentrations consistent with the labeled potency, and maintaining specified purity and dispersibility in simulated gastric and intestinal fluids without significant degradation of the attributes for which the stabilizer was employed. In some embodiments the stabilizer is an antioxidant, such as vitamin E. Other suitable antioxidants include hydroxytoluene, butyrate, quinone, ascorbic acid.

The composition formulation described herein may contain a preservative. Preservatives are compounds that inhibit microbial growth and are typically added to dispersions to prevent microbes from growing. Typical amounts of preservatives needed to pass anti-microbial effectiveness testing as described by USP and EU methodology are used to test appropriate preservative levels. Preservatives include but are not limited to potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Coloring agents provide coloration to the composition or dosage form. Such excipients can include food grade dyes and food grade dyes adsorbed onto a suitable adsorbent such as clay or aluminum oxide. The amount of the coloring agent can vary, for example from about 0.1 to about 5% by weight of the composition or from about 0.1 to about 1%.

The disclosure includes methods of using the said composition formulation as a dietary supplement for promoting healthy living in general, and for preventing and treating a patient in particular.

The disclosure includes methods of preventing and treating of cardiovascular-related diseases, autoimmune diseases, inflammatory diseases, central nervous system diseases, and cancers by providing the composition formulation of the present disclosure as described herein to a patient in need thereof.

Diseases and disorders that may be treated with the composition formulation of the present disclosure described herein include Cardiovascular disease and disorders include angina, atherosclerosis, hypercholesterolemia, hypertriglyceridemia, low HDL, high blood pressure, Raynaud's disease, and cardiac arrhythmias. The disclosure includes a method of preventing or reducing the risk of a second myocardial infarction by providing the said formulation as described herein at least one time per day for at least 60 days, 180 days, 360 days, or in perpetuity to a patient following a first myocardial infarction.

In one embodiment, the present disclosure provides a method of treating or preventing hereditary hypercholesterolemia and/or mixed dyslipidemia (Fredrickson Types IIa and IIb) in a subject in need thereof, comprising administration of one or more compositions as disclosed herein to the subject.

In another embodiment, the present disclosure provides a method of treating or preventing primary hypercholesterolemia and/or mixed dyslipidemia in a subject in need thereof, comprising administration of one or more compositions as disclosed herein to the subject.

In another embodiment, the present disclosure provides a method of treating or preventing risk of recurrent nonfatal myocardial infarction in a subject with a history of myocardial infarction, comprising administration of one or more compositions as disclosed herein to the subject.

In another embodiment, the present disclosure provides a method of treating, slowing progression of or promoting regression of atherosclerotic disease in a subject in need thereof, comprising administration of one or more compositions as disclosed herein to the subject.

In another embodiment, the present disclosure provides a method of inhibiting oxidation of lipoproteins in a subject in need thereof, comprising administration of one or more compositions as disclosed herein to the subject.

In another embodiment, the present disclosure provides a method of scavenging free radicals in a subject in need thereof, comprising administration of one or more compositions as disclosed herein to the subject.

In another embodiment, the present disclosure provides a method of treating or preventing very high serum triglyceride levels (e.g. Types IV and V hyperlipidemia) in a subject in need thereof, comprising administration of one or more compositions as disclosed herein to the subject.

In another embodiment, the present disclosure provides a method of treating or preventing rheumatic arthritis or chronic infectious arthritis, comprising administration of the compounded composition as disclosed herein to the subject.

In another embodiment, the present disclosure provides a method of enhance health, preventing diseases, and prolong life, comprising administration of the compounded composition as disclosed herein to the subject.

In another embodiment, the present disclosure provides a method of enhance health, preventing diseases, and prolong life to elder population, comprising administration of the compounded composition as disclosed herein to the subject.

In another embodiment, the present disclosure provides a method of treating and preventing constipation, comprising administrating low dose metformin in the composition formulation as disclosed herein to the subject.

It will be understood, however, that the specific dose level for any particular patient will depend upon a variety of factors including the activity of the specific compound employed, the age, body weight, general health, sex, diet, time of administration, route of administration and rate of excretion, drug combination and the severity of the particular disease undergoing therapy.

In one embodiment, a composition as described herein is administered to a subject once or twice or three times per day. In another embodiment, 1, 2, 3, 4, 5, 6, 7, 8 capsules, each containing specified combinations of composition active ingredients as described herein, are administered to a subject each time daily. In another embodiment, 1 or 2 or more capsules, each containing specified combinations of composition active ingredients as described herein, are administered to the subject in the morning, for example between about 5 am and about 11 am, and 1 or 2 or more capsules, each containing specified combinations of omega-3 plus active ingredients as described herein, are administered to the subject in the evening, for example between about 5 pm and about 11 pm.

In another embodiment, 1, 2, 3, 4, 5, 6, 7, 8 capsules, each containing specified combinations of the composition active ingredients as described herein, are administered to a subject each time daily. In another embodiment, the said capsules containing individual active ingredient can be taken simultaneously or at different times. In another embodiment, 1 or 2 or more capsules, each containing individual active ingredients as described herein, are administered to the subject in the morning, for example between about 5 am and about 11 am, and 1 or 2 or more capsules, each containing individual active ingredients as described herein, are administered to the subject in the evening, for example between about 5 pm and about 11 pm. The combination of the capsules comprises all of the active ingredient needed by the subject.

In another embodiment, the composition formulations useful in accordance with methods of the disclosure are orally deliverable. The terms “orally deliverable” or “oral administration” herein include any form of delivery of a therapeutic agent or a composition thereof to a subject wherein the agent or composition is placed in the mouth of the subject, whether or not the agent or composition is swallowed. Thus “oral administration” includes buccal and sublingual as well as esophageal administration. In one embodiment, the composition is present in a capsule, for example a soft gelatin capsule.

A composition for use in accordance with the disclosure can be formulated as one or more dosage units. The terms “dose unit” and “dosage unit” herein refer to a portion of a pharmaceutical composition that contains an amount of an omega-3 plus therapeutic agent suitable for a single administration to provide a therapeutic effect. Such dosage units may be administered one to a plurality (i.e. 1 to about 10, 1 to 8, 1 to 6, 1 to 4 or 1 to 2) of times per day, or as many times as needed to elicit a therapeutic response.

### DETAILED DESCRIPTION OF THE DISCLOSURE

The following examples provide illustration but not limit the present disclosure. The unit dosage in the following illustration table is the amount of active ingredient used each time. Specific dosage forms can be softgel, hard capsule, microcapsule powder, injectable, and any other forms of dosage that can be applied. Half a unit, 2 units, 3 units, or multi-units can be used each day. When multi-units are used, the units can be used separately, but can also be used simultaneously.

### Example 1: Compounded Composition

The formulation listed in Example 1 comprises capsules containing metformin, Omega-3 fatty acids, statin, and vitamins. For the application herein, metformin and statin can be administrated as tablets separately. The basic manufacturing procedure has been described herein and is within the skill in the art.

TABLE 1
Compounded composition-1: Metformin, Omega-3
fatty acids, Statin and Vitamins

| | | |
|---|---|---|
| Components | Unit Dose | Daily Dose (2 units) |

| | | | | |
|---|---|---|---|---|
| Omega-3 | 1000 | mg | 2000 | mg |
| Vitamin D3 | 400 | IU | 800 | IU |
| Vitamin B3(Niacin) | 20 | mg | 40 | mg |
| Vitamin E | 2 | mg | 4 | mg |
| Statin | 3 | mg | 6 | mg |
| Metformin | 125 | mg | 250 | mg |

### Example 2: Compounded Composition

The formulation listed in Example 2 comprises capsules containing metformin, Omega-3 fatty acids, statin, and vitamins. For the application herein, metformin and statin can be administrated as tablets separately. The basic manufacturing procedure has been described herein and is within the skill in the art.

TABLE 2
Compounded composition-2: Metformin, Omega-3
fatty acids, Statin and Vitamins

| | | |
|---|---|---|
| Components | Unit Dose | Daily Dose (2 units) |

| | | | | |
|---|---|---|---|---|
| Omega-3 | 1000 | mg | 2000 | mg |
| Vitamin D3 | 600 | IU | 1200 | IU |
| Vitamin B3(Niacin) | 40 | mg | 80 | mg |
| Vitamin E | 2 | mg | 4 | mg |
| Statin | 6 | mg | 12 | mg |
| Metformin | 250 | mg | 500 | mg |

### Example 3: Compounded Composition

The formulation listed in Example 3 comprises capsules containing metformin, Omega-3 fatty acids, statin, and vitamins. For the application herein, metformin and statin can be administrated as tablets separately. The basic manufacturing procedure has been described herein and is within the skill in the art.

TABLE 3
Compounded composition-3: Metformin, Omega-3
fatty acids, Statin and Vitamins

| | | |
|---|---|---|
| Components | Unit Dose | Daily Dose (2 units) |

| | | | | |
|---|---|---|---|---|
| Omega-3 | 1000 | mg | 2000 | mg |
| Vitamin D3 | 800 | IU | 1600 | IU |
| Vitamin B3(Niacin) | 60 | mg | 120 | mg |
| Vitamin E | 2 | mg | 4 | mg |
| Statin | 9 | mg | 18 | mg |
| Metformin | 250 | mg | 500 | mg |

### Example 4: Compounded Composition

The formulation listed in Example 4 comprises enteric capsules containing metformin, Omega-3 fatty acids, red yeast rice, and vitamins. For the application herein, metformin can be administrated as tablets separately. The basic manufacturing procedure has been described herein and is within the skill in the art.

TABLE 4
Compounded composition-4: Metformin, Omega-3
fatty acids, Red Yeast Rice and Vitamins

| | | |
|---|---|---|
| Components | Unit Dose | Daily Dose (2 units) |

| | | | | |
|---|---|---|---|---|
| Omega-3 | 1000 | mg | 2000 | mg |
| Red Yeast Rice (1% statin) | 300 | mg | 600 | mg |
| Vitamin D3 | 1000 | IU | 2000 | IU |
| Vitamin B3(Niacin) | 80 | mg | 160 | mg |
| Vitamin E | 2 | mg | 4 | mg |
| Metformin | 125 | mg | 250 | mg |

### Example 5: Compounded Composition

The formulation listed in Example 5 comprises enteric capsules containing metformin, Omega-3 fatty acids, statin, and vitamins. For the application herein, metformin and statin can be administrated as tablets separately. The basic manufacturing procedure has been described herein and is within the skill in the art.

TABLE 5
Compounded composition-5: Metformin, Omega-3
fatty acids, Statin and Vitamins

| | | |
|---|---|---|
| Components | Unit Dose | Daily Dose (2 units) |

| | | | | |
|---|---|---|---|---|
| Omega-3 | 900 | mg | 1800 | mg |
| Vitamin D3 | 1200 | IU | 2400 | IU |
| Vitamin B3(Niacin) | 100 | mg | 200 | mg |
| Vitamin E | 2 | mg | 4 | mg |
| Statin | 6 | mg | 12 | mg |
| Metformin | 250 | mg | 500 | mg |

### Example 6: Compounded Composition

The formulation listed in Example 6 comprises enteric capsules containing metformin, Omega-3 fatty acids, statin, and vitamins. For the application herein, metformin and statin can be administrated as tablets separately. The basic manufacturing procedure has been described herein and is within the skill in the art.

TABLE 6
Compounded composition-6: Metformin, Omega-3
fatty acids, Statin and Vitamins

| | | |
|---|---|---|
| Components | Unit Dose | Daily Dose (2 units) |

| | | | | |
|---|---|---|---|---|
| Omega-3 | 1000 | mg | 2000 | mg |
| Vitamin D3 | 1200 | IU | 2400 | IU |
| Vitamin B3(Niacin) | 100 | mg | 200 | mg |
| Vitamin E | 2 | mg | 4 | mg |
| Statin | 3 | mg | 6 | mg |
| Metformin | 125 | mg | 2500 | mg |

### Example 7: Compounded Composition

The formulation listed in Example 7 comprises enteric capsules containing metformin, Omega-3 fatty acids, red yeast rice, and vitamins. For the application herein, metformin can be administrated as tablets separately. The basic manufacturing procedure has been described herein and is within the skill in the art.

TABLE 7
Compounded composition-7: Metformin, Omega-3
fatty acids, Statin and Vitamins

| | | |
|---|---|---|
| Components | Unit Dose | Daily Dose (2 units) |

| | | | | |
|---|---|---|---|---|
| Omega-3 | 1000 | mg | 2000 | mg |
| Red Yeast Rice (1% statin) | 300 | mg | 600 | mg |
| Vitamin D3 | 1200 | IU | 2400 | IU |
| Vitamin B3(Niacin) | 150 | mg | 300 | mg |
| Vitamin E | 2 | mg | 4 | mg |
| Metformin | 125 | mg | 250 | mg |

### Example 8: Compounded Composition

The formulation listed in Example 8 comprises enteric capsules containing metformin, Omega-3 fatty acids, statin, and vitamins. For the application herein, metformin and statin can be administrated as tablets separately. The basic manufacturing procedure has been described herein and is within the skill in the art.

TABLE 8
Compounded composition-8: Metformin, Omega-3
fatty acids, Statin and Vitamins

| | | |
|---|---|---|
| Components | Unit Dose | Daily Dose (2 units) |

| | | | | |
|---|---|---|---|---|
| Omega-3 | 1000 | mg | 2000 | mg |
| Vitamin D3 | 1200 | IU | 2400 | IU |
| Vitamin B3(Niacin) | 50 | mg | 100 | mg |
| Vitamin E | 2 | mg | 4 | mg |
| Statin | 3 | mg | 6 | mg |
| Metformin | 500 | mg | 1000 | mg |

### Example 9: Therapeutic Intervention

(1) Correcting serum hyperlipidemia. Madam A is a 57-years old woman with hypertriglyceridemia. After taking the dosage form illustrated in example 4 (2 units/day) for 24 days, her serum triglyceride level from higher than normal of 2.32 to normal level of 1.46 mmol/L. The serum triglyceride level of Madam B is 1.82 mmol/L, and after taking the dosage form illustrated in example 4 (2 units/day) for 2 months, her serum triglyceride level decreased to normal range (1.34); in addition, her low-density lipoprotein (LDL “bad” cholesterol) from higher (4.35 mmol/L, normal <4.14) decreased to normal range (3.68). After 1 month of taking the dosage form illustrated in example 4 (2 units/day), the serum triglyceride level of Mr. C decreased to normal level (1.49) from higher (1.92), and LDL level also decreased.

(2) Correcting hypertension. A 72 years old woman had hypertension but can not be corrected by many hypertensive drugs. After 2 weeks of taking the dosage form illustrated in example 4 (2 units/day), blood pressure decreased significantly, and returned to normal level in 3 weeks.

(3) Xerophthalmia. A 56 years old type 2 diabetes patient, who suffered from xerophthalmia for many years, and need to use eye drops almost constantly. After 1 month of taking the dosage form illustrated in example 4 (2 units/day), his symptom improved significantly, and slowly can live normally without eye drops.

(4) Muscae volitantes. A woman patient of 63 years old, suffering from muscae volitantes for more than 2 years. After 2 months of taking the dosage form illustrated in example 4 (1 units/day), her symptom disappeared completely.

(5) Nervous system disease. A woman patient of 59 years old, after a brain tumor operation, started to show memory problems, and was out of her sense some time. After 3 months of taking the dosage form illustrated in example 4 (1 units/day), her symptoms become better, can more clearly expressed herself, and disorientation phenomena disappeared.

(6) Habitual constipation. A woman of 62 years old, become habitual constipation 40 years ago after over-taking antibiotics. After taking the dosage form illustrated in example 4 (2 units/day), she had diarrhea for 2-3 days, and at day 4 return to normal, and no more constipation problem after that.

To sum up, although the foregoing disclosure has been described in some detail by way of illustration for purposes of clarity and understanding, it will be apparent to those skill in the art that certain changes and modifications may be practiced. Therefore, descriptions and examples should not be construed as limiting the scope of the disclosure, which is delineated by the appended claims.

What is claimed is:

1. A metformin compounded composition, comprising active ingredients including metformin, Omega-3 fatty acids, statin, and vitamins.

2. The metformin compounded composition of claim 1, wherein, by weight ratio, metformin:omega-3 fatty acids:statin=50-500:1000:1-300.

3. The metformin compounded composition of claim 2, wherein, by weight ratio, metformin: omega-3 fatty acids:statin=100-300:1000:1-10.

4. The metformin compounded composition of claim 1, wherein said vitamins comprise vitamin D and/or vitamin B and/or vitamin E.

5. The metformin compounded composition of claim 1, wherein said Omega-3 fatty acids comprise EPA and/or DHA.

6. The metformin compounded composition of claim 1, wherein said statin comprises any synthetic statin-containing chemical drugs.

7. The metformin compounded composition of claim 6, wherein one of the said statin-containing natural products is red yeast rice.

8. A pharmaceutical medicine, wherein the pharmaceutical drug is oral dosage form comprising the compounded composition of claim 1.

9. The pharmaceutical medicine of claim 8, wherein said oral dosage form is an oral capsule or tablet comprising 0.1 mg to 2 g of metformin compounded composition claim 1; or said oral dosage form is an oral capsule or tablet comprising 0.1 mg to 2 g of metformin compounded composition of claim 1 for individual use.

10. The method for preventing and/or treating cardiovascular diseases, diabetes, chronic inflammation, cancer, autoimmune diseases, eye diseases, nervous system diseases, and constipation, comprising: administrating an effective amount of a pharmaceutical medicine of claim 8.
